(19) United States
(12) Patent Application Publication
(10) Pub. No.: US 2007/0270961 A1
(43) Pub. Date: Nov. 22, 2007

(54) Spinal implant with deployable and retractable barbs

(75) Inventor: Joe W. Ferguson, Collierville, TN (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/410,805

(22) Filed: Apr. 25, 2006

(51) Int. Cl. A61F 2/44

(52) U.S. Cl. 623/17.11

(57) ABSTRACT

The present invention provides spinal implants and systems which may be used for fixing a portion of the spine, such as the cervical spine, to allow correction or healing. One such spinal implant includes a body having at least one cavity therein. A barb is disposed within the cavity, with the barb adapted to rotate about a support member between a retracted position and a deployed position. In this manner, the barb may be selectively moved between desired positions, including the retracted and deployed positions. The retracted position may be used during insertion, removal, or repositioning of the implant between two vertebral bodies. The deployed barb position may be used for encouraging the barbs to engage the vertebral bodies to provide additional stability, promote fusion between the implant and vertebral bodies, hold the implant relative to the vertebral bodies, and the like.

### BACKGROUND

The present invention relates generally to orthopedic implants used for correction of spinal injuries or deformities, and more specifically, but not exclusively, to spinal implants, systems, and methods of use and methods of manufacture thereof, for fixing a portion of the spine, such as the cervical spine, to allow correction or healing.

In the field of spinal surgery, it is known to place implants into vertebrae for a number of reasons, including (a) correcting an abnormal curvature of the spine, including a scoliotic curvature, (b) to maintain appropriate spacing and provide support to broken or otherwise injured vertebrae, and (c) perform other therapies on the spinal column.

Some treatments involve the removal of a disk, distraction of the disk space, and the insertion of an interbody device between two adjacent vertebrae. The interbody device, which may include an artificial disk, or a variety of fusion cages or other aids, typically are coupled to one or more of the vertebral bodies. This coupling in some cases involves fixed spikes which engage the end plates of the vertebral bodies. The fixed nature of the spikes usually requires that the disk space be over distracted to provide sufficient clearance for insertion of the interbody device. Further, the fixed spikes can be problematic in the event the interbody device must be removed, or repositioned. Improvements are desired.

### SUMMARY

The present invention provides spinal implants and systems which may be used for fixing a portion of the spine, such as the cervical spine, to allow correction or healing. The present invention further provides methods of use and methods of manufacture of the implants and systems. In one embodiment, a spinal implant of the present invention includes a body having at least one cavity therein. A barb is disposed within the cavity, with the barb adapted to rotate about a support member between a retracted position and a deployed position. This movement may include rotating the barb from the retracted position to the deployed position, and from the deployed position to the retracted position. In this manner, the barb may be selectively moved between desired positions, including the retracted and deployed positions. For example, the retracted position may be used during insertion, removal, or repositioning of the implant between two vertebral bodies. The deployed barb position may be used for encouraging the barbs to engage the vertebral bodies to provide additional stability, promote fusion between the implant and vertebral bodies, hold the implant relative to the vertebral bodies, and the like.

In some aspects, the barb is rotatably coupled to the support member so that the barb can rotate through a desired range of rotation. The rotation range may include, without limitation, at least about forty-five degrees (45°) of rotation between the retracted and deployed positions, about ninety degrees (90°) of rotation, or other rotational ranges. In some aspects, the support member extends through the cavity to provide an axis of rotation for the barb. The barb may further be positioned within the cavity so that the barb is disposed below a surface of the implant body when the barb is in the retracted position. The barb may have a variety of shapes within the scope of the present invention. In a particular embodiment, the barb includes a barb body having a rounded outer edge and a pointed end. The barb may, but need not be, substantially C-shaped. In other embodiments, the barb includes a tapered tip, which may be shaped as a blade, a pyramid, an angled edge, or the like.

In some aspects, the spinal implant includes a deployment device adapted to engage the barb within the body to cause the barb to rotate into the deployed position. The deployment device may have a variety of shapes and configurations within the scope of the present invention. For example, in one aspect the deployment device includes a rod that is received by a channel within the body, with the channel in communication with the cavity. In alternative aspects, the deployment device is removably coupled to the body to hold the barb in the deployed position; is disposed within the body to be generally orthogonal to the support member; and/or is positioned so that its removal from the body allows the barb to rotate from the deployed position towards the retracted position.

In some embodiments, the spinal implant includes a plurality of barbs and a plurality of cavities. In some aspects, each barb resides in a separate cavity. In a particular aspect, the barbs are adapted to rotate in a same direction when engaged by the deployment member or members. The barb axis of rotation may be below the surface of the implant body, and the barbs may each have separate support members, or some or all barbs may share one or more support members. In other aspects, the body includes an opening passing at least part way therethrough, with the opening adapted to receive a spinal implant placement instrument, a fixation element for coupling the body to the vertebral body, or the like.

In a particular embodiment of the present invention, a spinal implant includes a first body having a barb disposed within an aperture and adapted to rotate about a support member, a second body, and a nucleus member disposed between the first and second bodies. In this embodiment, the spinal implant may operate as an artificial disc, with the first and second bodies positioned adjacent, abutting or coupled to two opposing vertebral bodies. In some aspects, the nucleus is adapted to allow relative movement, such as relative rotational motion, between the first and second implant bodies.

The present invention further provides methods of using a spinal implant. In one embodiment, the method includes providing a spinal implant as described herein, inserting the implant between two vertebral bodies, and rotating the barb about the support member to cause the barb to engage one of the vertebral bodies. In one aspect, rotation of the barb includes inserting a deployment device into the spinal implant first body. The deployment device engages a barb body to rotate the barb from a retracted position to a deployed position. In one aspect, the method includes fixing the barb in the deployed position by coupling the deployment device to the spinal implant first body.

In one particular embodiment, the method includes retracting the spinal implant, with the retraction causing or helping cause the barb to rotate about the support member. For example, retracting the spinal implant may cause the barb to rotate from a deployed position to a retracted position. The retraction may include applying a translational force to the implant body, with the translational force applying a rotational force to the barb. In this manner, the retraction force on the implant helps cause or causes the barb to rotate out of the vertebral body and towards the retracted position.

Other features and advantages of the invention will appear from the following description in which the preferred embodiment has been set forth in detail in conjunction with the accompanying drawings.

### BRIEF DESCRIPTION OF THE DRAWINGS

FIG. 1A shows an overall view of a spinal implant according to one embodiment of the present invention;

FIG. 1B shows an exploded view of the spinal implant depicted in FIG. 1A;

FIG. 1C shows an end view of the spinal implant depicted in FIG. 1A;

FIGS. 1D-1E show cross-sectional views of the spinal implant depicted in FIG. 1A with the barbs in a deployed position and a retracted position, respectively;

FIG. 2A shows the spinal implant of FIG. 1A engaging a vertebral body;

FIG. 2B shows the spinal implant of FIG. 1A being distracted from a disk space;

FIG. 3A shows an overall view of a spinal implant according to another embodiment of the present invention;

FIG. 3B shows an exploded view of the spinal implant depicted in FIG. 3A;

FIG. 3C shows an end view of the spinal implant depicted in FIG. 3A;

FIG. 3D shows a cross-sectional view of the spinal implant depicted in FIG. 3A with the barbs in a deployed position;

FIG. 4A shows an overall view of a spinal implant according to an embodiment of the present invention;

FIG. 4B shows an exploded view of the spinal implant depicted in FIG. 4A;

FIG. 4C shows an end view of the spinal implant depicted in FIG. 4A;

FIG. 4D shows a cross-sectional view of the spinal implant depicted in FIG. 4A with the barbs in a deployed position;

FIG. 5A shows an overall view of a spinal implant according to one embodiment of the present invention;

FIG. 5B shows an exploded view of the spinal implant depicted in FIG. 5A;

FIG. 5C shows an end view of the spinal implant depicted in FIG. 5A;

FIG. 5D shows a cross-sectional view of the spinal implant depicted in FIG. 5A with the barbs in a deployed position;

FIG. 6 shows a simplified side view of a spinal implant according to another embodiment of the present invention disposed in a disk space;

FIG. 7 shows a simplified side view of a spinal implant according to an embodiment of the present invention; and

FIG. 8 is a simplified flow chart of a method of using a spinal implant according to an embodiment of the present invention.

### DETAILED DESCRIPTION

Reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of embodiments of the invention as illustrated therein, being contemplated as would normally occur to one skilled in the art to which the invention relates.

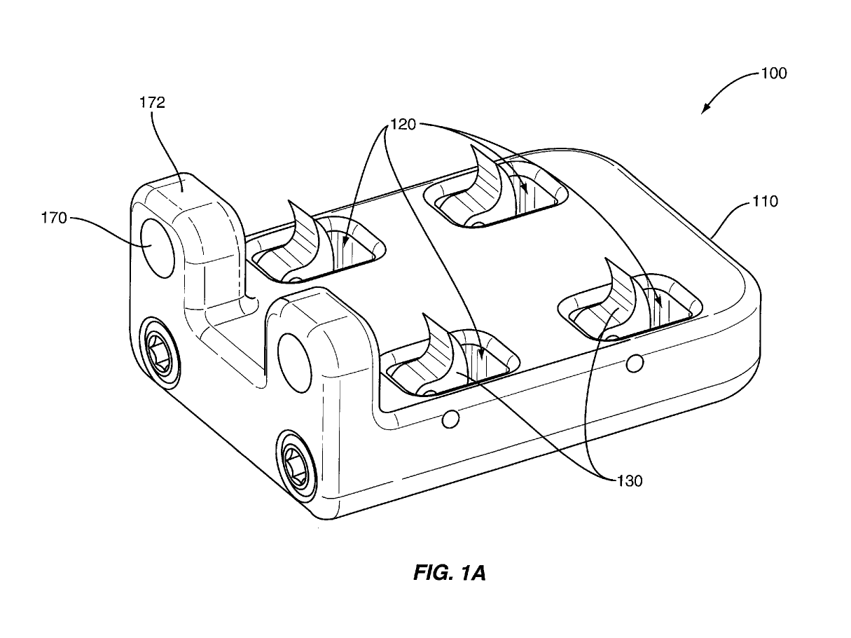

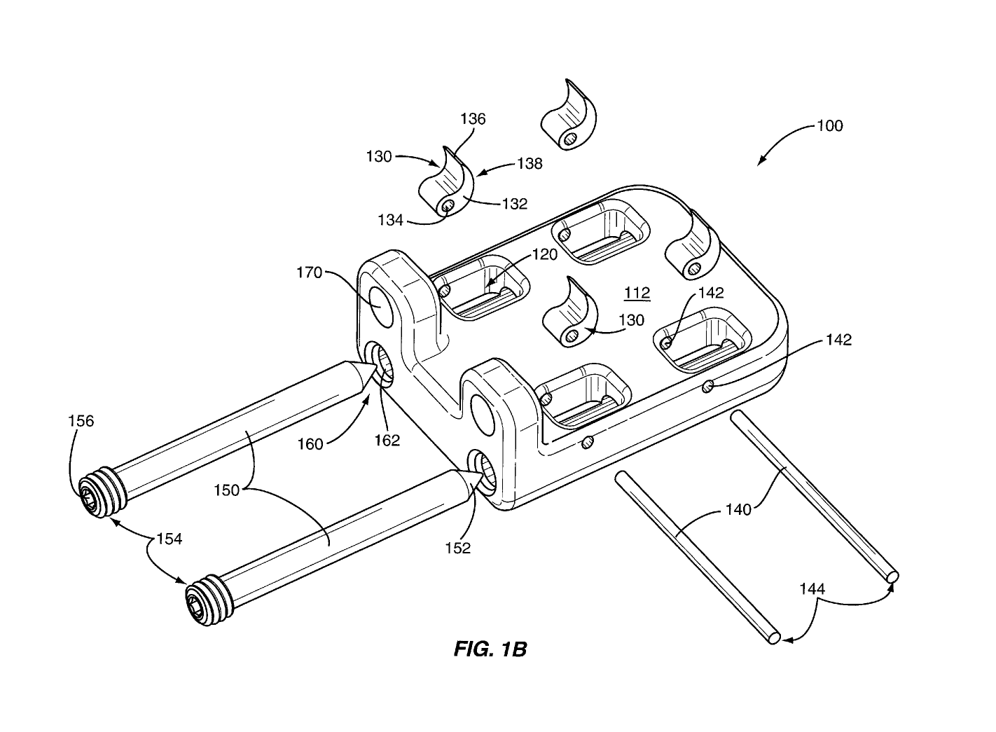

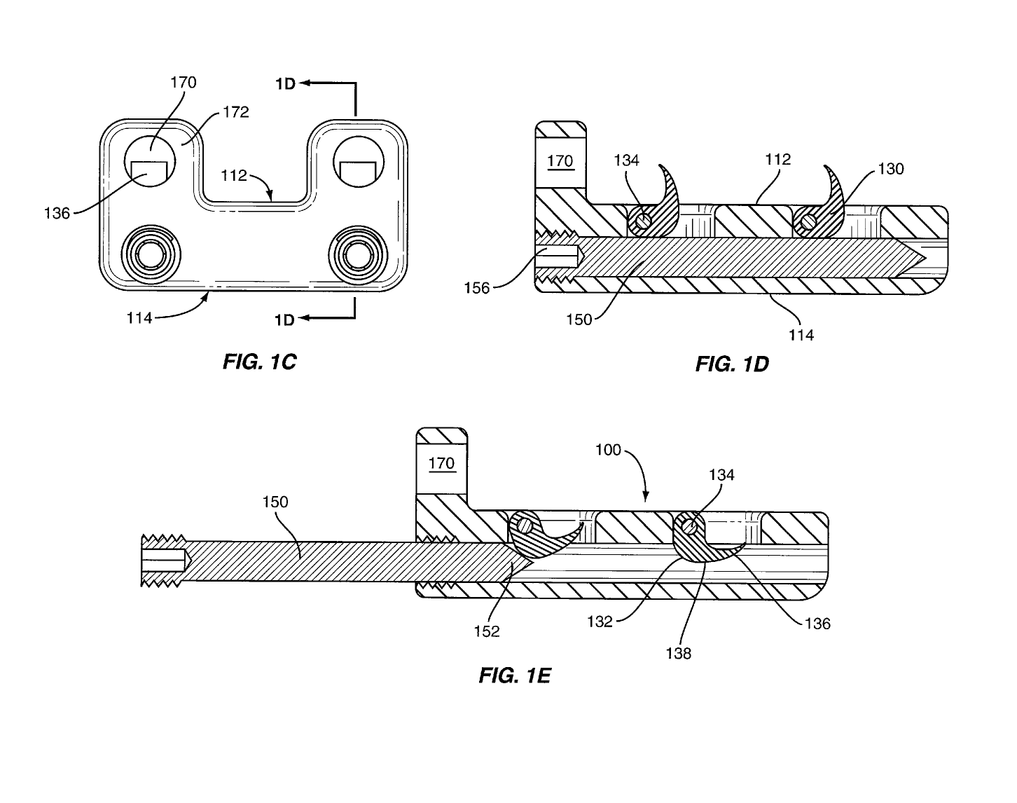

Turning to FIGS. 1A-1E, a spinal implant 100 according to an embodiment of the present invention will be described. Implant 100 includes a body 110 having spaced apart first and second surfaces 112, 114. As best seen in FIGS. 1A and 1B, one or more cavities 120 are formed in body 110. In a particular embodiment, cavities 120 are formed in first surface 112 and extend at least part way into body 110 towards second surface 114. Cavity 120 has a barb 130 disposed therein. In the depicted embodiment, implant 100 has four cavities 120, each containing one barb 130. In other embodiments, the number of cavities and barbs differ than depicted in the figures. For example, implant 100 may have fewer or greater numbers of cavities 120. While the figures depict each cavity 120 with a single barb 130, in other embodiments one or more cavities 120 have more than a single barb 130 disposed therein. In still other embodiments, one or more cavities 120 do not have any barbs 130. Implant 100 may have two (2), three (3), four (4), five (5), six (6), or more cavities 120, and a similar or different number of barbs 130.

In a particular embodiment, barb 130 is rotatably coupled to body 110, and in some embodiments, is rotatably coupled to a support member 140 within cavity 120. In this embodiment, barb 130 has an axis of rotation that is below first surface 112. Barb 130 axis of rotation may coincide with support member 140. This occurs, in one embodiment, when support member 140 passes through a hole 134 in a body portion 132 of barb 130. In a preferred embodiment, barb 130 rotates freely about support member 140. In this embodiment, support member 140 is inserted into a support member channel 142, and locked in place at or near a support member end 144. Support member 140 may be locked or otherwise coupled to body 110 using a weld, an adhesive, a mechanical lock, or another mechanism or technique. With support member 140 coupled to body 110, barb 130 may be rotated about support member 140.

In other embodiments, barb 130 is rotatably coupled to the inside of cavity 120. This may occur, for example, by providing barb body 132 with extensions, prongs, or the like (not shown), that engage a detent, gap, hole or the like in opposing walls of cavity 120. In one embodiment, the barb body extensions are integrally formed with barb body 132 so that barb body 132 does not need hole 134. In this embodiment, the extensions fit into the opposing detents, cavities, gaps, holes, or the like within cavity 120 walls so that the extensions rotate freely therein. In this manner, barb body 132 can rotate within cavity 120 to permit barb 132 to move between retracted and deployed positions. Other devices and methods for rotatably coupling barb 130 to implant body 110 also fall within the scope of the present invention.

In one embodiment, barb(s) 130 are adapted to rotate between a deployed position and a retracted position. Barbs 130 are depicted in a deployed position in FIGS. 1A, 1C and 1D. Barbs 130 are shown in a retracted position in FIG. 1E. Barbs 130 are moved between the retracted position and deployed positions using, in some embodiments, a deployment member 150. In a particular embodiment, deployment member 150 is inserted into a channel 160 in body 110 to engage one or more barbs 130. In this embodiment, channel 160 is in communication with one or more cavities 120. The embodiment depicted in FIG. 1 shows two deployment members 150, with each deployment member 150 engaging two barbs 130. Other embodiments of the present invention include different numbers and combinations of deployment members 150 and barbs 130.

In one embodiment, deployment member 150 is an elongate member having a shaped distal end 152 which is inserted into implant body 110. The elongate deployment member 150 may have a cross sectional shape which coincides with the shape of channel 160. The depicted embodiment shows generally cylindrical elongate deployment members having a circular cross section received by similar shaped channels 160. In other embodiments, deployment member 150 may have elliptical, square, rectangular or other cross sectional shapes. Shaped end 152 is adapted to engage barb 130 to rotate barb 130 from the retracted position to the deployed position. In a particular embodiment, shaped end 152 is angled, pointed, conical, chamfered, or the like to engage barb 130, and more particularly, to engage barb body 132 as best seen in FIG. 1E. Once shaped distal end 152 contacts barb body 132, the continued insertion of deployment member 150 into channel 160 rotates barb 130 into the position depicted in FIG. 1D. For example, deployment member 150 shaped distal end 152 engages barb body 132, causing barb 130 to rotate about support member 140 or to otherwise rotate within cavity 120.

In some embodiments, deployment member 150 has a lock mechanism 156 disposed at or adapted to be coupled to the deployment member 150 proximal end. Lock mechanism 156 may comprise a variety of mechanisms or techniques to couple deployment mechanism 150 to barb body 110, including without limitation a screw, a pin with a C-clip, a pin with a cap, a cam lock, and the like. In a particular embodiment, deployment member 150 includes a threaded proximal end 154 which engages with a threaded opening 162 of channel 160 to secure deployment member 150 to implant body 110. Threaded proximal end 154 may include a locking device, such as a cap, C-clip, set screw, or the like, to prevent or help prevent deployment member 150 from backing out of channel 160.

In one embodiment, spinal implant 100 includes one or more extensions or flanges 172 extending from body 110. Extensions 172 may be used, for example, to engage a posterior or anterior surface of the vertebral body to which spinal implant 100 is coupled. In some embodiments, extension 172 has an opening 170. Opening 170 may extend at least part way through extension 172. In this manner, opening 172 can be a tool-engaging recess, adapted to receive a spinal implant placement or revision instrument. The instrument, for example, may engage opening 172 to hold spinal implant body 110 during surgery. In another embodiment, opening 170 passes completely through extension 172. In this embodiment, opening 170 is adapted to receive a spinal implant placement or revision instrument, or to receive a fixation element for coupling body 110 to a vertebral body. The fixation element may comprise a wide range of devices, including vertebral bone screws or the like. The size, shape and number of extensions 172, and openings 170 therein, may vary within the scope of the present invention compared to that depicted in the Figures.

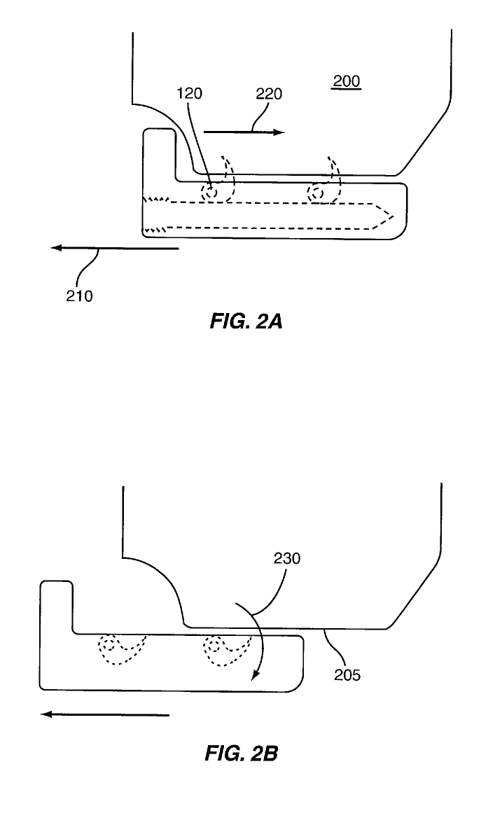

With reference to FIGS. 1A-1E, 2A and 2B, a use of one embodiment of implant 100 will be described. In this embodiment, implant 100 is inserted into a disk space between two vertebral bodies, with FIGS. 2A and 2B depicting only one of the vertebral bodies 200 for ease of illustration. Preferably, implant 100 is inserted into the disk space when barbs 130 are in the retracted position (FIG. 1E). In a particular embodiment, barbs 130 are disposed below first surface 112 during implant 100 insertion. In this manner, barbs 130 do not damage the vertebral body during implant insertion. Additionally, the disk space does not need to be overdistracted to prevent barbs 130 from damaging the vertebral body since barbs 130 are in a retracted position, and may be below first surface 112. After insertion of implant 100, barbs 130 are moved to the deployed position (FIG. 1D) to engage the vertebral body. This may occur, for example, using deployment member 150 as described herein to rotate barbs 130 to engage an end plate 205 of vertebral body 200. In one embodiment, barbs 130 engage the cancellous bone of vertebral body 200. Barbs 130 are affixed in the deployed position, such as by coupling deployment member 150 to implant body 110. The implant may be further coupled to vertebral body 200 using fixation devices, screws, or the like, which again may couple extensions 172 of implant body 110 to vertebral body 200.

In the event spinal implant 100 is to be removed or repositioned, it may be desirable to have barbs 130 disengage from vertebral body 200. In a particular embodiment of the present invention, deployment member 150 is partially or fully removed from implant body 110 to permit barbs 130 to return to the retracted position. The removal of deployment member 150, in some embodiments, occurs prior to or contemporaneously with the removal of implant 100. In a preferred embodiment, barbs 130 are permitted to rotationally move from the deployed position to the retracted position. Barbs 130, in some cases, may rotate to the retracted position after removal of deployment member 150. This may occur, for example, in the event spinal implant 100 has been recently implanted, too recent for substantial bony growth which may engage implant 100 and/or barbs 130.

In other embodiments, as shown in FIGS. 2A and 2B, the removal of deployment member 150 facilitates the rotation of barbs 130 to the retracted position upon the application of a desired force to barb 130. For example, the application of a translation force to implant body 110, as shown by arrow 210, causes an opposite translation force to be applied to barbs 130 as shown by arrow 220. The application of force 220 to barbs 130 causes barbs 130 to rotate within cavities 120 towards the retracted position as shown by arrow 230. In this manner, pulling on or otherwise moving implant body 110 causes or helps cause barbs 130 to rotate out of vertebral body 200 towards the retracted position. As a result, barbs 130 do not significantly damage vertebral body 200, or endplate 205, as may occur with prior art devices having fixed spikes. If desired, implant 100 may be repositioned between vertebral bodies 200, and barbs 130 redeployed.

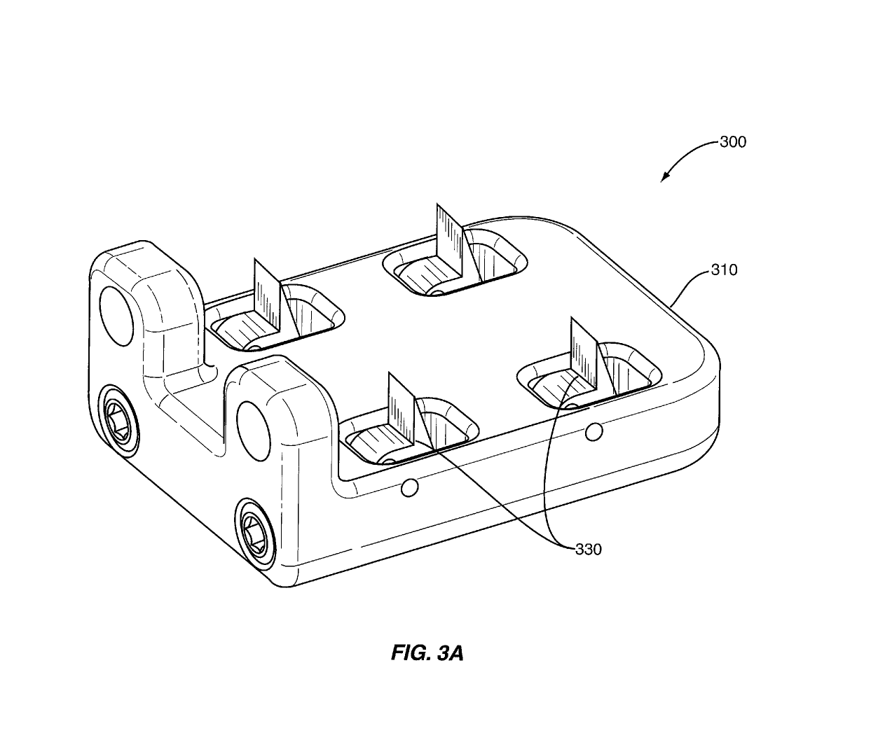

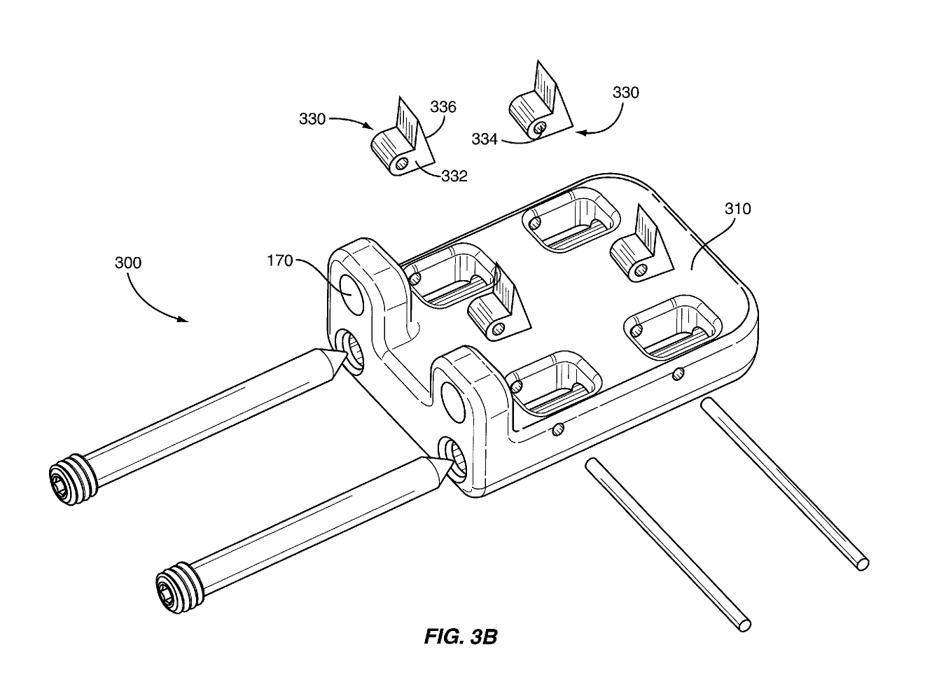

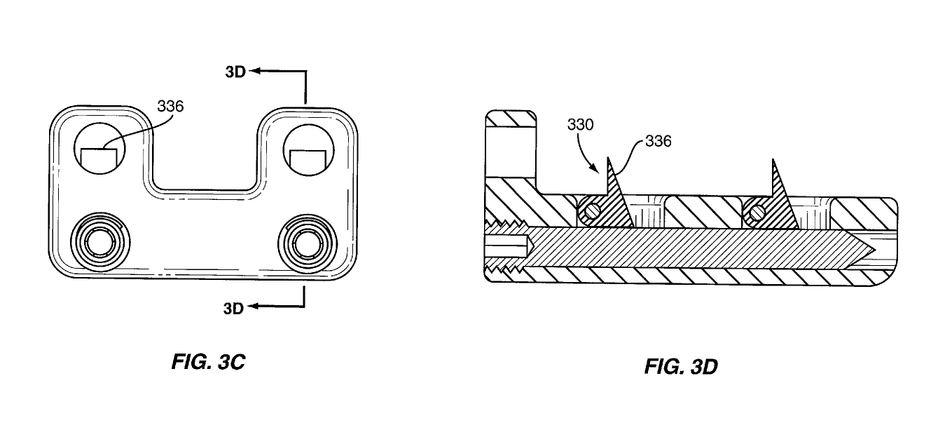

It will be appreciated by those skilled in the art that implant 100 depicted in FIGS. 1A-2B is representative of a number of different embodiments that fall within the scope of the present invention. FIGS. 3A-3D depict still another embodiment of the present invention. Many of the components and characteristics of this embodiment are similar to those described in conjunction with FIGS. 1 and 2, and will not be repeated. This embodiment, however, uses a different barb or spike configuration. As seen in FIGS. 3A-3D, spinal implant 300 includes one or more barbs 330 having a different shape than barbs 130. In one embodiment, barb 330 includes a barb body 332 and a tapered tip 336 extending therefrom. In some embodiments, barb body 332 has a hole 334 disposed part way or fully therethrough, to facilitate a rotational coupling between barb 330 and an implant body 310. In this embodiment, tapered tip 336 has a blade edge shape. Blade edge 336 rotates into and out of endplates of the vertebral body in a manner similar to that described with the curved barb tips of FIGS. 1 and 2.

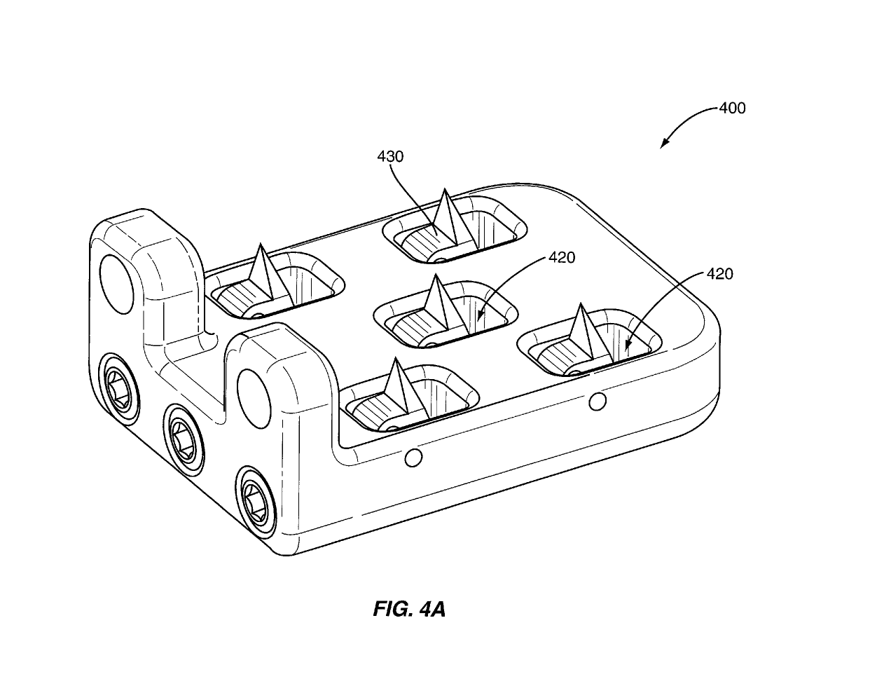

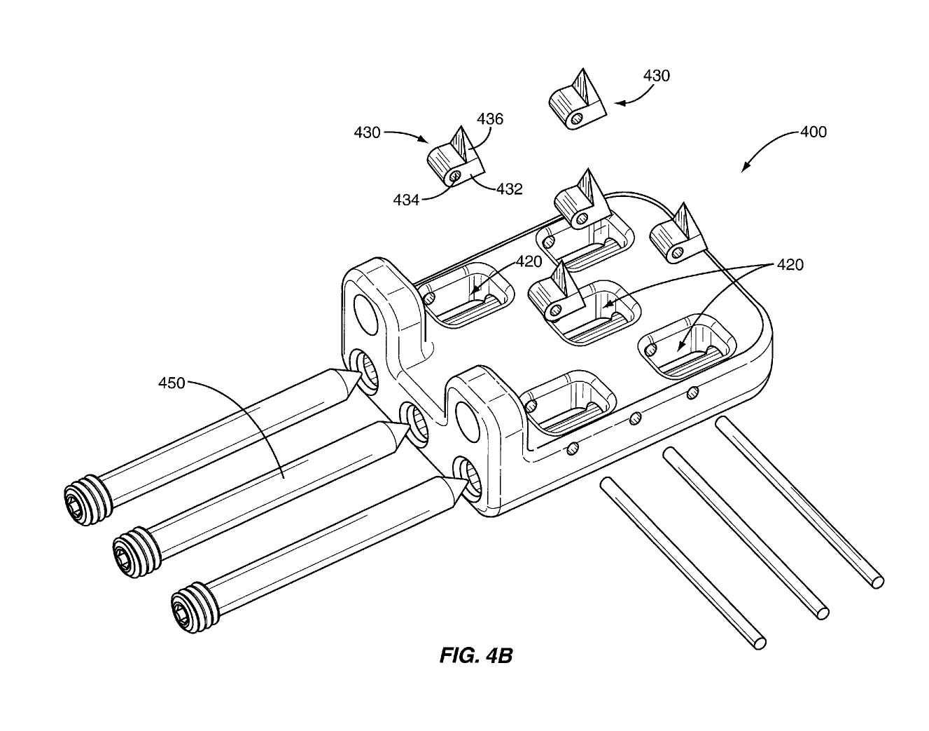

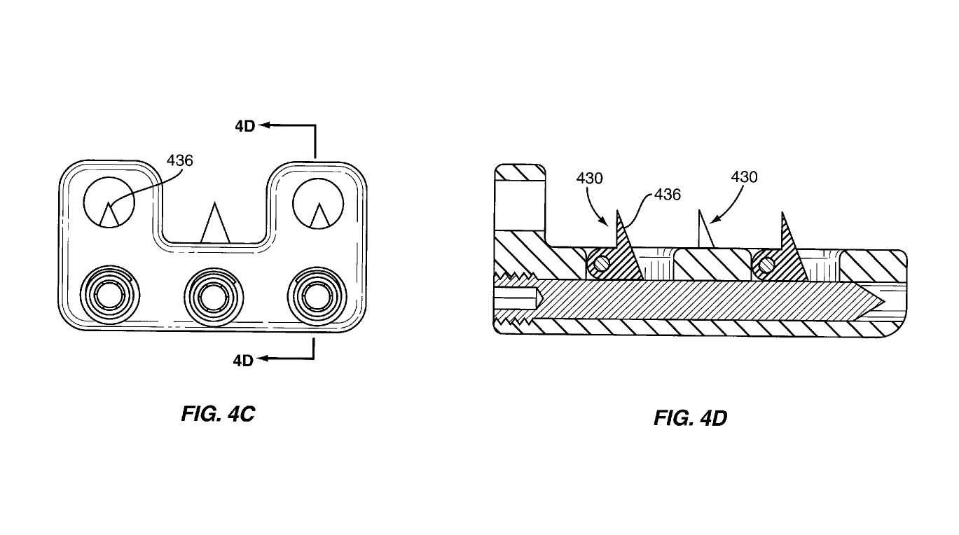

FIGS. 4A-4D depict an alternative embodiment of the present invention. Again, many of the features and characteristics of spinal implant 400 depicted in FIG. 4 are similar or identical to those described in conjunction with earlier embodiments. Implant 400, however, includes a different barb configuration 430. In particular, barb 430 includes a barb body 432 having a tapered tip 436 in the shape of a diamond or pyramid. Barb body 432 may include a hole or other mechanism 434 for coupling barb body 432 to an implant body 410, to an implant cavity 420, and/or to a support member 440. Tapered tip 436 rotates into the endplate of the vertebral body to help secure implant 400 thereto. In a particular embodiment, as best shown in FIGS. 4A-4C, implant 400 includes five (5) cavities 420 each with a single barb 430 contained therein. Barbs 430 are rotated from a retracted position to a deployed position using three (3) deployment members 450. It will be appreciated by those skilled in the art that fewer or greater numbers and combinations of cavities 420, barbs, 430 and/or support members 440 may be used within the scope of the present invention.

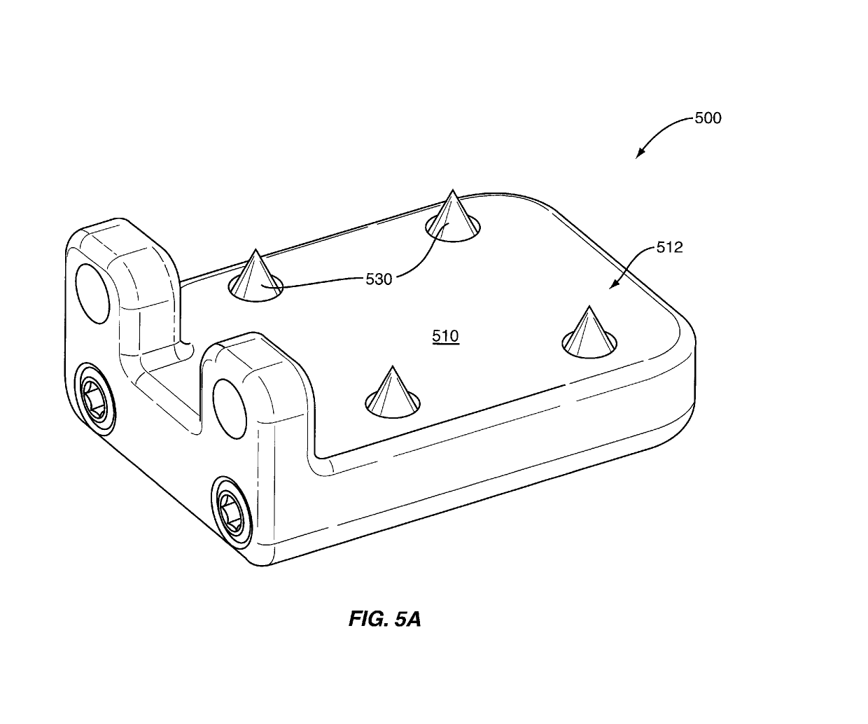

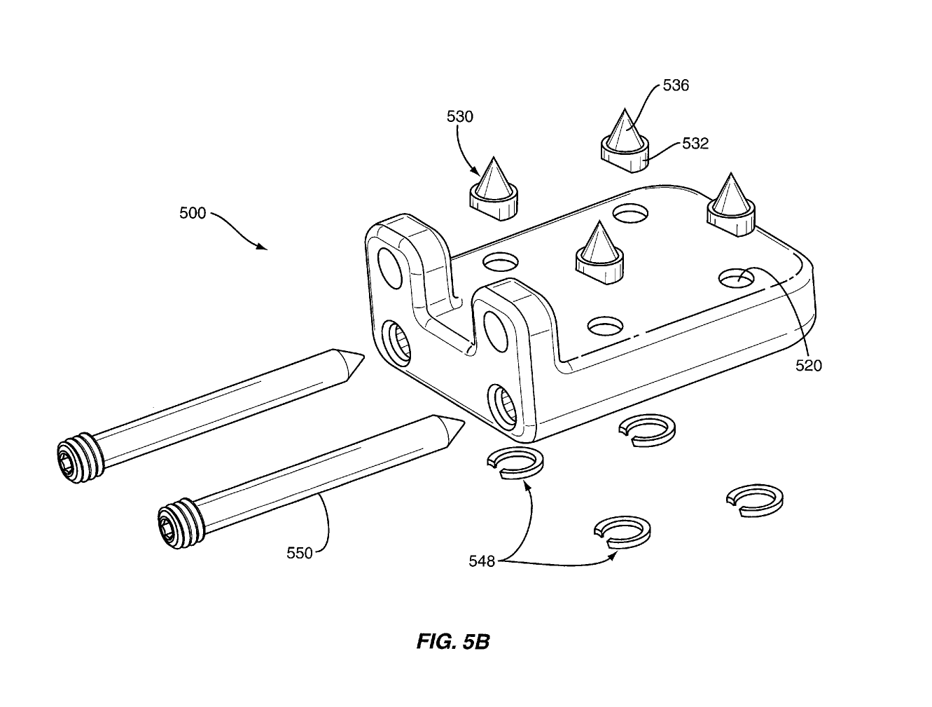

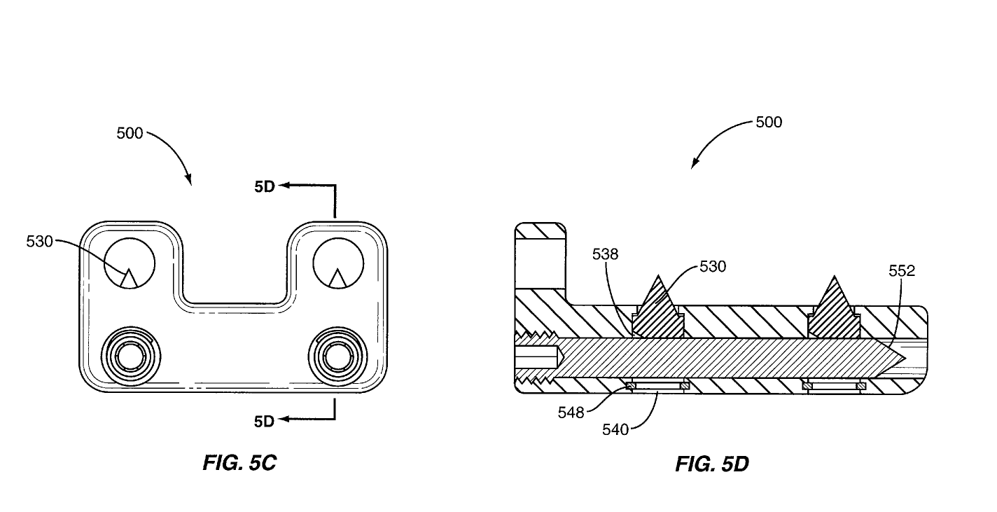

FIGS. 5A-5D depict still another embodiment of the present invention. In this embodiment, a spinal implant 500 having an implant body 510 includes one or more barbs 530. Barbs 530 are adapted to translate above a surface 512 of spinal implant body 510 to engage a vertebral body (not shown). Barbs 530 include a barb body 532 and a tip 536. Barbs 530 extend above surface 512 by passing at least part way through an aperture 520 in body 510. As best shown in FIGS. 5B and 5D, implant 500 includes a deployment member 550 having a tapered tip 552 which engages a lower edge 538 of barb 530. In one embodiment, lower edge 538 of barb 530 is a chamfered edge 538 having an angle generally corresponding to the angle of tapered tip 552. This arrangement allows for the smooth deployment of barb 530 from a retracted position to a deployed position. Implant 500 further includes one or more holes or channels 540 in communication with apertures 520. Channels 540 are adapted to receive barbs 530. A lock mechanism 548 engages one end of barb 530, and/or channel 540 to prevent barb 530 from exiting implant body 510. Again, the arrangement and number of barbs 530, and the combination of barbs 530, apertures 520 and deployment members 550, may vary from that depicted in the figures.

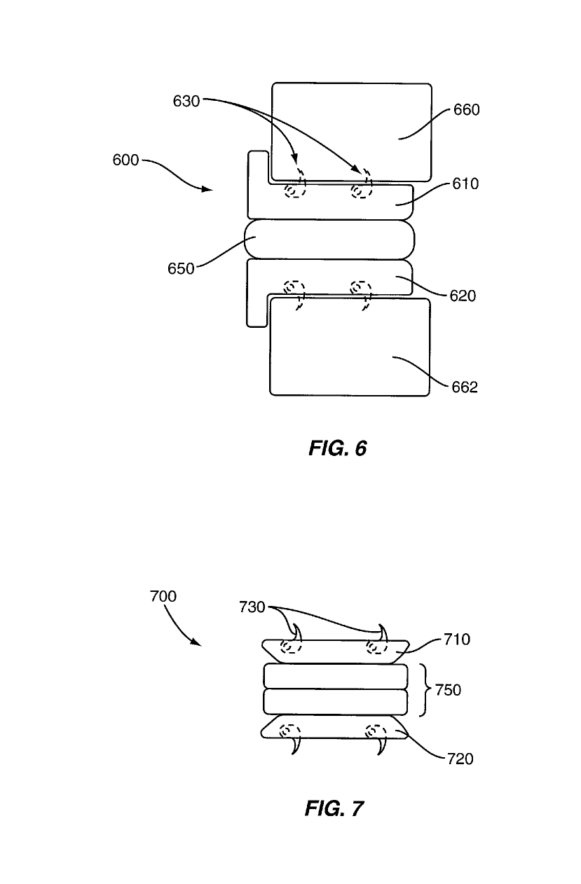

FIG. 6 depicts an alternative embodiment of the present invention. In this embodiment, a spinal implant 600 comprises an artificial disc-like device having two members 610 and 620 each adapted to engage a vertebral body. One or both members 610, 620 have one or more barbs 630 adapted to be deployed therefrom. In one embodiment, barb(s) 630 corresponding to member 610 are adapted to engage a superior vertebral body 660 while barb(s) 630 corresponding to member 620 are adapted to engage an inferior vertebral body 662. Barbs 630 from member 610 and/or member 620 may be deployable as described in conjunction with FIGS. 1A-5D. In some embodiments, members 610 and 620 correspond to one or more of the spinal implant embodiments described in conjunction with FIGS. 1-5. In other embodiments, only one of member 610 or member 620 includes barbs 630. Barbs 630 may be selectively deployed, and retracted, to help provide secure attachment of implant 600 and facilitate the repositioning or removal of implant 600 as needed.

In the embodiment of FIG. 6, implant 600 further includes a nucleus portion 650 disposed between first and second members 610 and 620. Nucleus portion 650, which may comprise a wide range of materials and components, is adapted to allow relative movement between first member 610 and second member 620. Nucleus portion 650 may comprise a metal, a plastic, a ceramic, or other materials such as polyethylene, or the like. In a preferred embodiment, nucleus portion 650 allows relative rotational movement between first and second members 610, 620. In this manner, deployable barbs are used with spinal implants designed to allow increased flexibility for the patient compared to spinal fusion plates, implants, and the like.

In one embodiment, nucleus portion 650 is integrally formed with or comprises part of first member 610 and/or second member 620. For example, first and second members 610, 620 may form a ball and trough arrangement similar to that disclosed in U.S. Pat. No. 6,113,637, entitled “Artificial Intervertebral Joint Permitting Translational and Rotational Motion,” the complete disclosure of which is incorporated herein by reference. First member 610 may define the ball and second member 620 may define the trough, or vice versa. In this manner, interfacing surfaces of first and second members 610, 620 provide the means for articulation of implant 600. Further, in some embodiments, barbs 630 may be used instead of or in addition to screw fixation to help couple implant 600 to one or more vertebral bodies. First and second members 610, 620 may also define a ball and socket, or other configuration, with the interface of the two members providing for relative movement therebetween. Other means for articulation between members 610, 620 also fall within the scope of the present invention.

FIG. 7 depicts a spinal implant 700 according to another embodiment of the present invention. Implant 700 generally comprises a three component articulating device having first and second endplates 710 and 720, disposed around a core element 750. In some embodiments, core element comprises a medical grade plastic, and endplate 710, 720 comprise a medical grade metal. In one embodiment, endplate 710 and/or endplate 720 includes deployable protrusions 730 adapted to engage the vertebral bodies. In a particular embodiment, the protrusions 730 are rotatably deployed according to one or more of the methods and systems described herein.

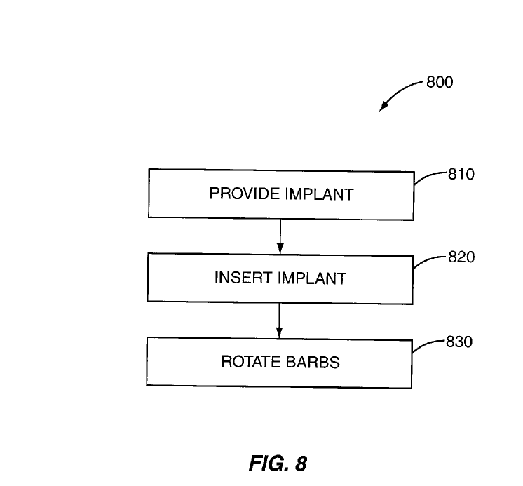

FIG. 8 depicts a simplified schematic of a method 800 for using a spinal implant according to one embodiment of the present invention. As shown, method 800 includes providing a spinal implant (Block 810). The spinal implant may be any of the implant embodiments described herein. In particular, the spinal implant includes at least one barb disposed within a cavity of the implant and adapted to rotate at least part way out of the cavity. The method includes inserting the spinal implant between two vertebral bodies (Block 820) and rotating the barb to cause the barb to engage one of the vertebral bodies (Block 830). In this manner, the barb can be in a retracted position when the spinal implant is inserted into the patient, and subsequently rotated or otherwise deployed into the vertebral body to provide an additional fixation device for securing the implant to the patient vertebral body.

Components of the described embodiments may be made from a variety of materials compatible for use with the human body, including without limitation metals (e.g., titanium, nitinol, stainless steel), ceramics, polyethylene, PEEK, and other materials.

Having described several embodiments, it will be recognized by those skilled in the art that various modifications, alternative constructions, and equivalents may be used without departing from the spirit of the invention. Accordingly, the above description should not be taken as limiting of the scope of the present invention.

What is claimed is:

1. A spinal implant, comprising:
a body having at least one cavity therein; and
a barb disposed within the cavity, the barb adapted to rotate about a support member from a retracted position to a deployed position, and from the deployed position to the retracted position.

2. The spinal implant as in claim 1 wherein the support member extends through the cavity to provide an axis of rotation for the barb.

3. The spinal implant as in claim 1 wherein the barb rotates through at least about forty-five degrees (45°) of rotation between the retracted and deployed positions.

4. The spinal implant as in claim 1 wherein the barb rotates through about ninety degrees (90) of rotation between the retracted and deployed positions.

5. The spinal implant as in claim 1 wherein the body has spaced apart first and second surfaces, the first surface having the cavity formed therein, and wherein the barb is disposed below the first surface when in the retracted position.

6. The spinal implant as in claim 1 further comprising a deployment device adapted to engage the barb within the body to cause the barb to rotate into the deployed position.

7. The spinal implant as in claim 6 wherein the deployment device comprises an elongate member that is received by a channel within the body, the channel in communication with the cavity.

8. The spinal implant as in claim 6 wherein the deployment device is removably coupled to the body to hold the barb in the deployed position.

9. The spinal implant as in claim 6 wherein the deployment device is disposed within the body to be generally orthogonal to the support member.

10. The spinal implant as in claim 6 wherein the deployment device is disposed within the body at an angle relative to the support member.

11. The spinal implant as in claim 1 wherein removal of the deployment device from the body allows the barb to rotate from the deployed position towards the retracted position.

12. The spinal implant as in claim 1 wherein the barb comprises a barb body having a rounded outer edge and a pointed end.

13. The spinal implant as in claim 12 wherein the barb body is substantially C-shaped.

14. The spinal implant as in claim 1 wherein the barb comprises a barb body and a tapered tip.

15. The spinal implant as in claim 14 wherein the tapered tip is pyramidal in shape.

16. The spinal implant as in claim 14 wherein the tapered tip is a blade tip.

17. The spinal implant as in claim 14 wherein the barb body comprises a hole adapted to receive the support member.

18. The spinal implant as in claim 1 wherein the barb is adapted to engage a vertebral body when in the deployed position.

19. The spinal implant as in claim 1 wherein the barb is one of a plurality of barbs and the cavity is one of a plurality of cavities.

20. The spinal implant as in claim 19 wherein the plurality of barbs are adapted to rotate in a same direction when rotated from the deployed position to the retracted position.

21. The spinal implant as in claim 19 wherein the plurality of barbs are adapted to rotate in opposite directions when rotated from the deployed position to the retracted position.

22. The spinal implant as in claim 1 wherein the body further comprises an opening passing at least part way therethrough, the opening adapted to receive a spinal implant placement instrument.

23. The spinal implant as in claim 1 wherein the body further comprises a hole passing therethrough, the hole adapted to receive a fixation element for coupling the body to a vertebral body.

24. A spinal implant, comprising:
a body having at least one cavity therein;
a plurality of barbs, at least one of the plurality of barbs disposed within the at least one cavity; and
a deployment member adapted to engage at least one of the plurality of barbs to move the barb from a retracted position to a deployed position;
wherein the plurality of barbs move in a same direction when moved from the retracted position to the deployed position.

25. A spinal implant, comprising:
a first body adapted to be disposed adjacent a first vertebral body, the first body having at least one aperture therein with a first barb disposed within the aperture, the first barb adapted to rotate about a support member;
a second body adapted to be disposed adjacent a second vertebral body; and
a nucleus member disposed between the first and second bodies.

26. The spinal implant as in claim 25 wherein the first and second bodies are adapted to be coupled to opposing first and second vertebral bodies in a disc space.

27. The spinal implant as in claim 25 wherein the nucleus is adapted to allow relative movement between the first and second bodies.

28. The spinal implant as in claim 25 wherein a barb deployment device is removably coupled to the first body to rotate the first barb between a retracted position and a deployed position.

29. The spinal implant as in claim 28 wherein the barb deployment device is disposed within the first body to be generally orthogonal to the support member.

30. The spinal implant as in claim 28 wherein the barb deployment device is disposed within the first body oriented at an acute angle relative to the support member.

31. The spinal implant as in claim 28 wherein removal of the barb deployment device from the first body allows the first barb to rotate from the deployed position towards the retracted position.

32. The spinal implant as in claim 25 wherein the second body further comprises at least one aperture having a second barb disposed therein, the second barb adapted to rotate about a second support member.

33. The spinal implant as in claim 25 wherein the first body has spaced apart first and second surfaces, the first surface having the aperture formed therein, and wherein the first barb is disposed within the first body below the first surface when the first barb is in a retracted position.

34. The spinal implant as in claim 33 wherein an axis of rotation of the first barb is below the first surface.

35. The spinal implant as in claim 25 wherein the first barb comprises a substantially C-shaped barb body having a rounded outer edge and a pointed end.

36. A spinal implant, comprising:
a first body adapted to be disposed adjacent a first vertebral body, the first body having at least one aperture therein with a first barb disposed within the aperture, the first barb adapted to move relative to the aperture to engage the first vertebral body;
a second body adapted to be disposed adjacent a second vertebral body; and
means for articulating the first and second bodies.

37. The spinal implant as in claim 36 wherein the means for articulating the first and second bodies comprises a first articulation surface of the first body, the first articulation surface in at least partial contact with a second articulation surface of the second body.

38. The spinal implant as in claim 36 wherein the means for articulating the first and second bodies comprises a ball and trough relationship of a portion of the first and second bodies.

39. The spinal implant as in claim 36 wherein the means for articulating the first and second bodies includes an intermediate member that is positioned between the first body and the second body.

40. A method of using a spinal implant, the method comprising:
providing a spinal implant comprising a first body having at least one cavity therein, and a barb disposed within the cavity, wherein the barb is adapted to rotate about a support member from a retracted position to a deployed position, and from the deployed position to the retracted position;
inserting the spinal implant between two vertebral bodies; and
rotating the barb about the support member to cause the barb to engage one of the vertebral bodies.

41. The method as in claim 40 wherein the rotating of the barb comprises inserting a deployment device into the spinal implant first body, the deployment device engaging a barb body to rotate the barb from the retracted position to the deployed position.

42. The method as in claim 41 further comprising fixing the barb in the deployed position by coupling the deployment device to the spinal implant first body.

43. The method as in claim 40 further comprising retracting the spinal implant, wherein retracting the spinal implant causes the barb to rotate about the support member.

44. The method as in claim 43 wherein retracting the spinal implant causes the barb to rotate from the deployed position to the retracted position.

45. The method as in claim 43 wherein retracting the spinal implant comprises applying a translational force to the implant body, the translational force applying a rotational force to the barb.

46. The method as in claim 40 wherein the spinal implant further comprises a second body and a nucleus member disposed between the spinal implant first and second bodies.

47. The method as in claim 40 wherein the spinal implant further comprises a second body in articulating relationship with the first body.
